(19) United States
(12) Patent Application Publication
(10) Pub. No.: US 2007/0112366 A1
(43) Pub. Date: May 17, 2007

(54) Apparatus and method for releasing tendon sheath

(76) Inventors: Kenneth M. Welborn, Charlottesville, VA (US); Joseph F. Slade III, Guilford, CT (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/272,052

(22) Filed: Nov. 14, 2005

(51) Int. Cl. A61B 17/32

(52) U.S. Cl. 606/170

(57) ABSTRACT

An apparatus and method are disclosed for release of a tendon sheath using a rasp tool and an endoscopic cutting tool. The rasp tool has a body supporting a probe with a rasp surface at one end of the probe for removing soft tissue adhering to the tendon sheath after insertion of the probe into a pocket formed above the tendon sheath. The body, rasp probe and rasp surface are formed in one piece and may be mounted removably on an endoscope. The endoscopic cutting tool has a probe with a blade at one end, the blade being extendable by operation of a trigger after insertion of the probe into the pocket, the blade being operable to cut the tendon sheath by pulling the tool out of the pocket with the blade extended. The trigger is part of a pistol grip assembly that is mounted on the cutting blade probe and endoscope so that it can be rotated about the axis of the cutting blade probe. The endoscope illumination can be provided with ultraviolet light to make visible objects not visible otherwise.

### BACKGROUND OF THE INVENTION

1. Field of the Invention

The present invention generally relates to techniques for alleviating pain in the movement of limbs and, in particular, to techniques for releasing tendon sheaths where pain is due to inflammation of tissue within the sheath.

2. Background Description

In 1895, Fritz de Quervain, a Swiss surgeon, first described tenosynovitis (inflammation of the tendons) within the first dorsal wrist extensor compartment (tendon tunnel on the dorsal wrist surface) at the radial styloid (base of thumb). The usual complaint was pain in the region of the wrist joint. De Quervain's tenosynovitis is a painful and often disabling condition that is mainly observed in workers, athletes and musicians who perform repetitive manual tasks.

De Quervain tenosynovitis (sometimes also called de Quervain's tendinitis) is a result of friction of the tendon as it glides through narrow channels of the first dorsal compartment found along the thumb side of the wrist. This compartment contains abductor pollicis longus (APL) and extensor pollicis brevis (EPB) tendons. These channels or fascia tubes guide the tendons to their specific point of insertion where they act to position the wrist or fingers to accomplish a specific goal.

Anatomic variation within the first extensor compartment has been identified. One author (Harvey, F J, Harvey P M, Horsley M W. “De Quervain's disease: surgical or nonsurgical treatment”, Journal of Hand SurgeryI, 1990; vol. 15, pp. 83-87) found a separate synovial compartment containing the extensor pollicis brevis at surgery in 91% of cases. There are also reports of a separate compartment for the EPB (Harvey F J 1990). Cadaveric dissection studies have shown that this variant is present in 40% of wrists (Witt J, Pess G, Gelberman R H. “Treatment of de Quervain tenosynovitis”, Journal of Bone Joint Surgery, 1991; vol. 73, pp. 219-222).

The friction or resistance to tendon gliding results in injury which is manifested as inflammation and swelling of the tendons and compartment. With increasing inflammation local tissues begin to swell and tendon gliding becomes increasingly difficult with greater resistance to gliding and greater injury. Setting up an increasing exponential injury cycle which must be broken before pain relief can be achieved.

The superficial branch of the radial nerve crosses over the first dorsal compartment and is susceptible to both inflammation and injury. Injury to this nerve often results in severe pain syndrome (Regional Causalgia) which is sympathetically mediated. Local inflammation leads to swelling and in time the surrounding structures also become swollen and irritated including the Superficial Branch of the Radial Nerve which lies directly over the tendon sheath. Additional Injury to this nerve can result from increased inflammation, traction and disruption which greatly complicate treatment and delay functional recovery.

Swelling can cause pain and tenderness along the thumb side of the wrist, usually noticed when forming a fist, grasping or gripping things, or turning the wrist. Pain over the thumb side of the wrist is the main symptom. The pain may appear either gradually or suddenly. It is felt in the wrist and can travel up the forearm. The pain is usually worse with use of the hand and thumb, especially when forcefully grasping things or twisting the wrist. Swelling over the thumb side of the wrist is noticed and may be accompanied by a fluid-filled cyst in this region. There may be an occasional “catching” or “snapping” when moving the thumb. Because of the pain and swelling, it may be difficult to move the thumb and wrist, such as in pinching. Irritation of the nerve lying on top of the tendon sheath may cause severe pain and numbness on the back of the thumb and index finger.

Risk for these injuries is apparent in people employed in work requiring repetitive use of their hands. Upper extremity work-related diagnoses are becoming more frequent as a source of chronic pain and lost work time for the injured worker. De Quervain's tendinitis is reported to be one of the most common disorders reported by working people in the United States. Extensive epidemiological investigation indicates that the adverse ergonomic exposures of force, repetition, vibration and certain postures are risk factors for development these disorders. Annual incidence of hand and arm tendinitis from computer, data entry and keyboard use has been measured at 39 cases/100 person-years. The most common disorder was deQuervain's tendinitis. More than 50% of computer users reported tendinitis during the first year after starting a new job.

Treatment for the above described disorders is directed at decreasing tendon swelling and nerve irritation, thereby relieving pain caused by tendon and nerve irritation and swelling. Early treatment includes splinting, therapy and non-steriodal medication, resting by splinting the thumb and wrist, and anti-inflammatory medication. Injection of corticosteroid into the tendon compartment may help reduce the swelling and relieve the pain. One source reported 40% failure with single injection requiring multiple injections. These injections are not without possible complications. Cheiralgia paresthetica, a mononeuropathy of the superficial branch of the radial nerve, usually results from local trauma to the wrist. One report describes subdermal atrophy following local hydrocortisone injection, and also describes linear atrophy which traverse the superficial radial nerve and contribute to the symptoms.

When symptoms are severe or do not improve, surgery may be recommended. The surgery opens the compartment covering, called the extensor retinaculum, which tightly secures the tendons against the radial styloid, to make more room for the irritated tendons. The surgeon then moves aside other tissues and locates the tendons and the tunnel. An incision is made to split the roof, or top, of the tunnel. This allows the tunnel to open up, creating more space for the tendons. The skin is then stitched together, and the hand is wrapped in a bulky dressing. The tunnel will eventually heal closed, but it will be larger than before. Scar tissue will fill the gap where the tunnel was cut.

However, traditional open surgical release has the potential for additional soft tissue injury. What is needed is minimally invasive surgery using an endoscopic technique to reduce local injury and avoid complications permitting a more rapid decline in symptoms.

### SUMMARY OF THE INVENTION

It is therefore an object of the present invention to provide an endoscopic technique for release of tendon sheaths.

A further object of the invention is to provide a technique for release of tendon sheaths that is minimally invasive.

Yet another object of the invention is to provide supporting tools that make a technique for release of tendon sheaths reliable and routine.

The invention provides a rasp tool and and endoscopic cutting tool for release of tendon sheath, and in particular for use in release of tendon sheath in treatment of de Quervain's tenosynovitis. An aspect of the invention is a kit comprising a rasp tool having a body supporting a probe with a rasp surface at one end of the probe for removing soft tissue adhering to the tendon sheath after insertion of the probe into a pocket formed above the tendon sheath, and an endoscopic cutting tool having a probe with a blade at one end, the blade being extendable by operation of a trigger after insertion of the probe into the pocket, the blade being operable to cut the tendon sheath by pulling the tool out of the pocket with the blade extended. The rasp body and probe may be fitted over an endoscope, a field of view for an endoscope camera being provided by a groove on the rasp surface. Further, the rasp body, rasp probe and rasp surface may be of one piece, the one piece being removably attachable to the endoscope.

In a further aspect of the invention, an endoscopic cutting tool is provided for use in release of tendon sheath, comprising an endoscope having a body from which is extended an endoscope tube with a camera lens at a tube end away from the body, and a probe with a blade at one end, the probe being mounted on the endoscope tube so that the blade end extends beyond the end of the tube to allow for extension and retraction of the blade, the blade being extendable by operation of a trigger, the trigger being operated after insertion of the probe into a pocket formed above the tendon sheath, the blade being operable to cut the tendon sheath by pulling the tool out of the pocket with the blade extended. In another aspect of this invention, the trigger is part of a pistol grip assembly mounted on the endoscopic cutting tool, the assembly being able to rotate around the axis of the probe. The invention further a light source and fiber optic channels for delivering light from the light source along the endoscopic tube to illuminate objects observable by the camera lens. In one aspect of the invention the light source provides ultraviolet light.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is an isometric view of the hand showing the thumb tendons and covering sheath.

FIG. 2A is a cross sectional cutaway view of the wrist showing the compartment containing the thumb tendons and a pocket cleared by a rasp in accordance with the invention. FIG. 2B is an expanded view from FIG. 2A of the portion of the wrist involving the thumb tendons.

FIG. 3A is a side view of the rasp used to clear a pocket in accordance with the invention. FIGS. 3B and 3C are sectional and perspective views, respectively, of the rasp instrument.

FIG. 4A is a side view of a pistol grip cutting tool used to release the tendon sheath. FIGS. 4C and 4E are perspective and sectional views, respectively, of the tool shown in FIG. 4A. FIG. 4B shows the tool with its cutting blade extended. FIGS. 4D and 4F are perspective and sectional views, respectively, of the tool with blade extended as shown in FIG. 4B.

### DETAILED DESCRIPTION OF A PREFERRED EMBODIMENT OF THE INVENTION

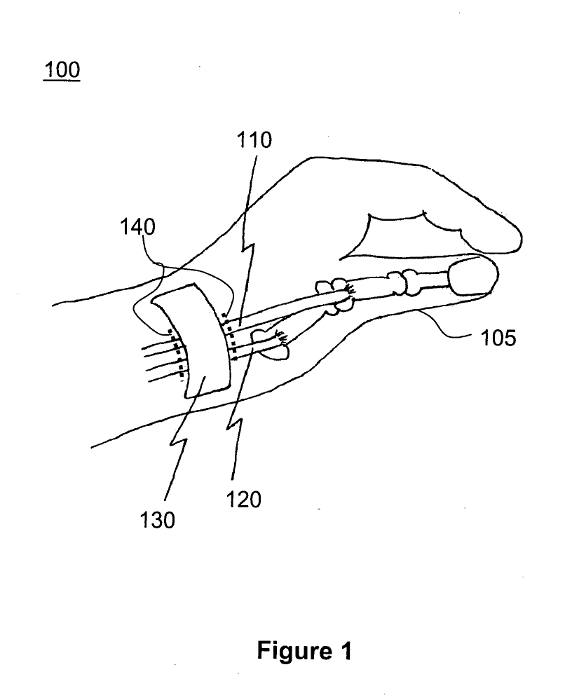

Referring now to the drawings, and more particularly to FIG. 1, there is shown a schematic drawing of hand 100, with particular attention to the thumb 105 and the two thumb tendons, the abductor pollicis longus (APL) 120 and extensor pollicis brevis (EPB) 110 tendons. Also shown is a portion of the upper side 130 of the sheath (the extensor retinaculum) holding the thumb tendons in place as they pass from the radius bone of the forearm along the inside edge of the wrist. The object of the instrument combination described herein and the methodology for using the instruments is to release the sheath 130 by cutting across the sheath between the two dotted lines 140.

An incision is made on the proximal or distal side of the sheath 130 and, after dissection down to the sheath 130 and then dissection across the sheath to create a pocket above the first dorsal compartment, a rasp tool is used to clear the tissue that adheres to the surface of the first dorsal compartment.

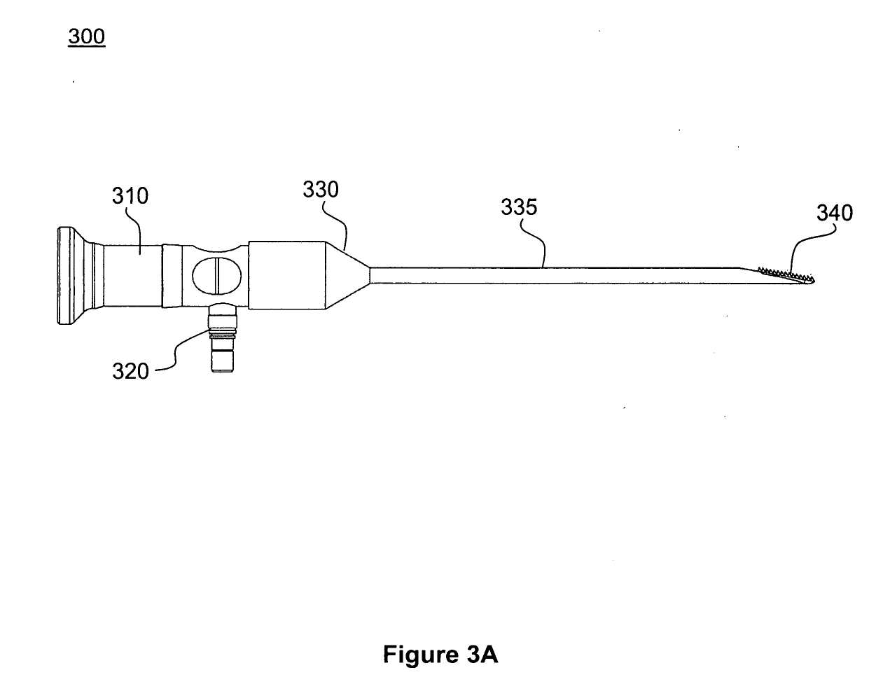

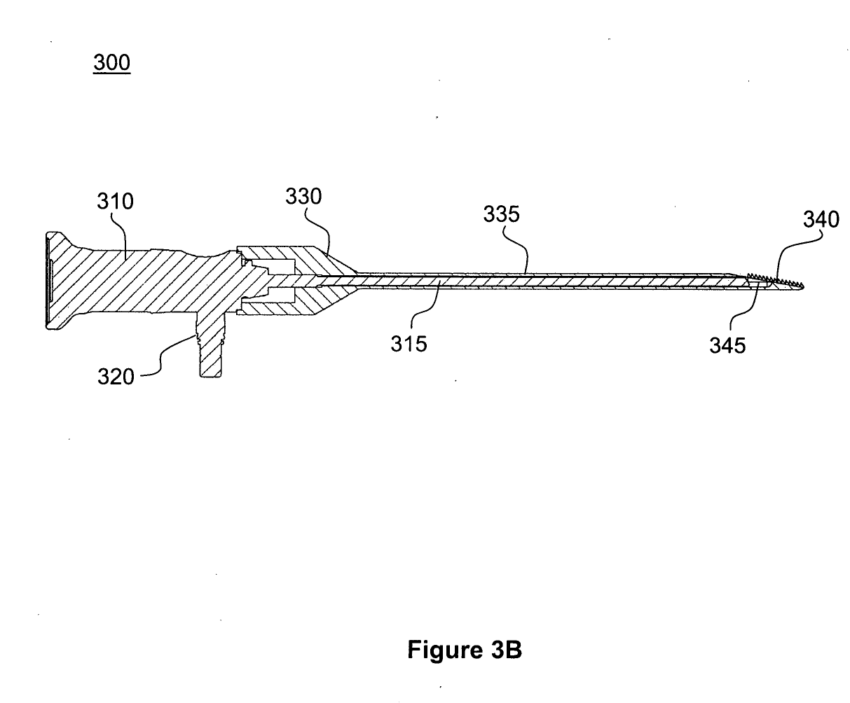

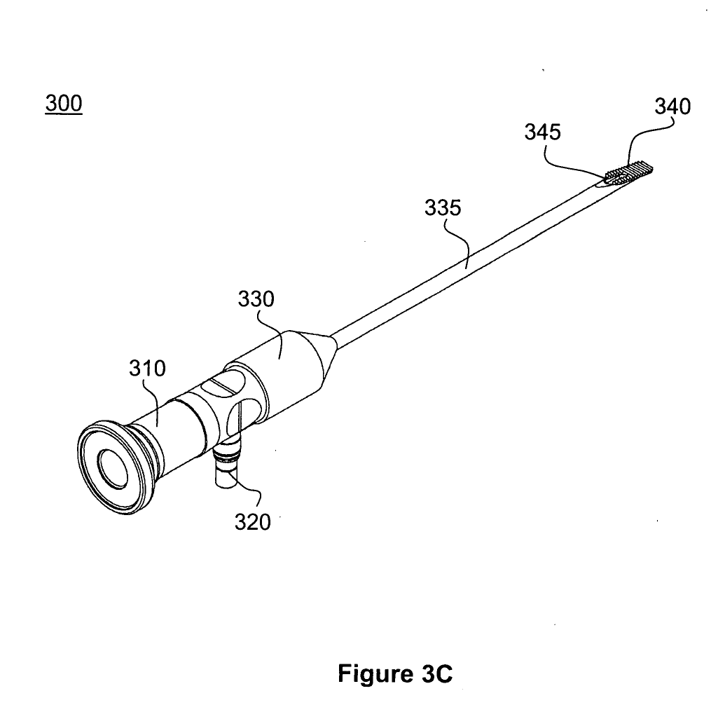

The rasp tool is shown in FIG. 3A. A sectional view of the rasp tool is shown in FIG. 3B, and a perspective view is shown in FIG. 3C. An endoscope is shown, having a handle 310 and endoscopic tube 315 to which is attached a removable a rasp 330 having a hollow probe 335 which fits conformably around endoscopic tube 315. At the end of the probe 335 is a rasp surface 340 disposed at a slight angle. At the end of endoscopic tube 315 is a camera lens, and there is a groove 345 in the rasp surface 340 so that the camera lens will have an unobstructed field of view. The light for the endoscope is provided by fiber optic channels (not shown) within the endoscopic tube 315. The light source is provided through endoscopic control 320, which also channels the video signals coming from the camera lens.

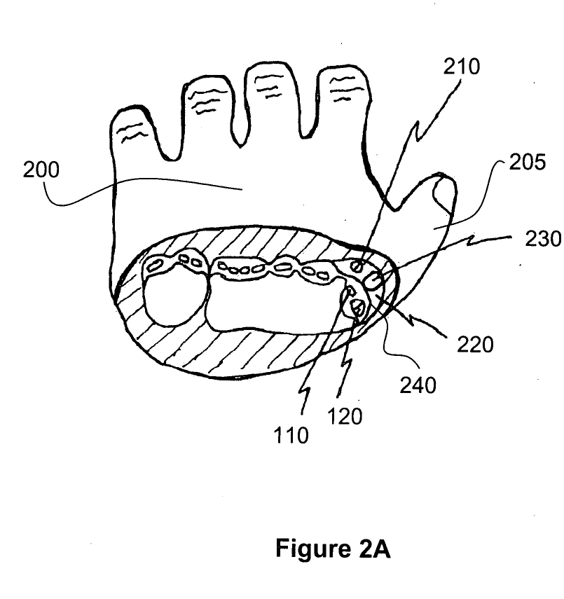

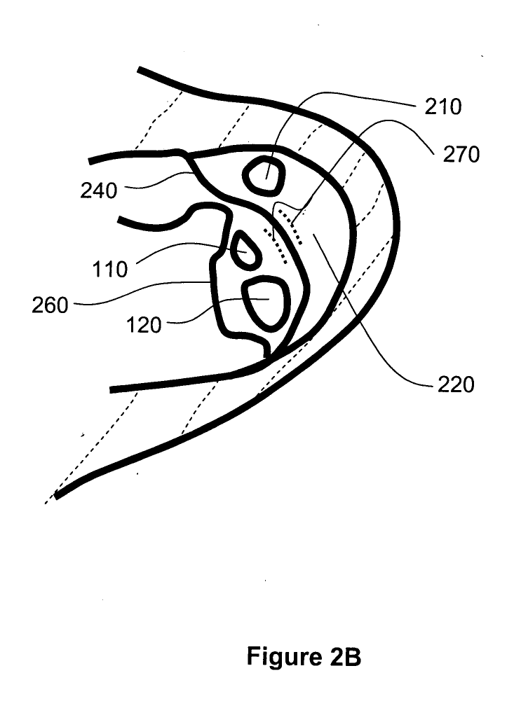

A cross sectional view of the hand 200 at the wrist is shown in FIG. 2A, with an expanded view of the portion of the wrist containing the thumb tendons shown in FIG. 2B. The thumb tendons (EPB 110 and APL 120) are held within a tunnel formed between an upper sheath surface 240 and a lower sheath surface 260. The pocket 220 above the upper sheath 240 contains a nerve 210 (the superficial branch of the radial nerve), which must be avoided. The rasp tool is inserted into the incision along the pocket 220, with the rasp surface 340 oriented so that the rasp surface 340 faces the upper sheath surface 240 from direction of the pocket 220. The rasp tool 300 is manipulated with the visual aid provided by the endoscope 310 to remove the soft tissue adhering to the outer surface 240 of the first dorsal compartment. Tissue removal will be complete when the operator of the rasp tool 300 detects a gritty or rough surface sensation, indicating abrasion against the surface of the sheath 240.

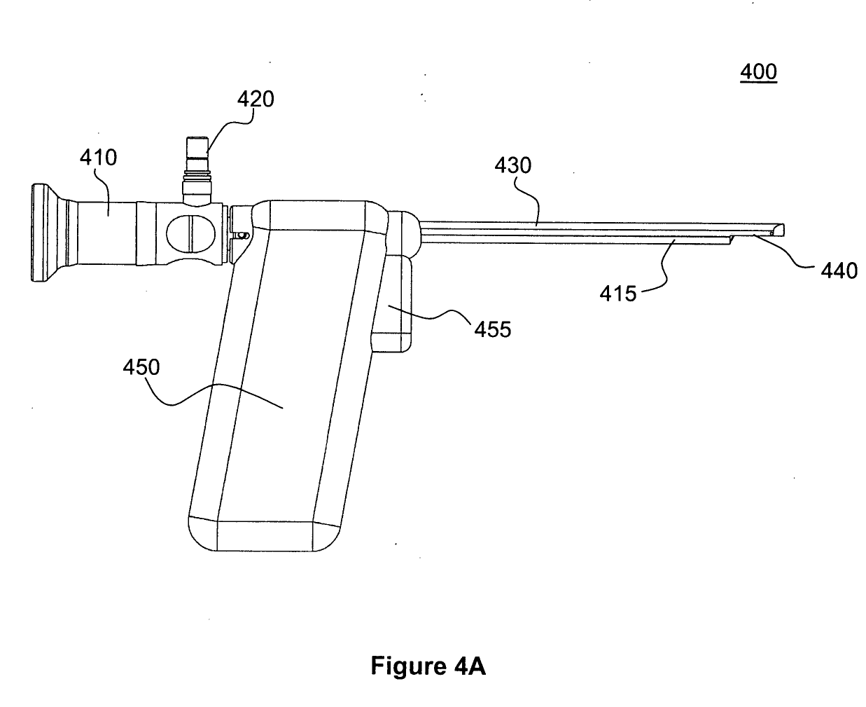

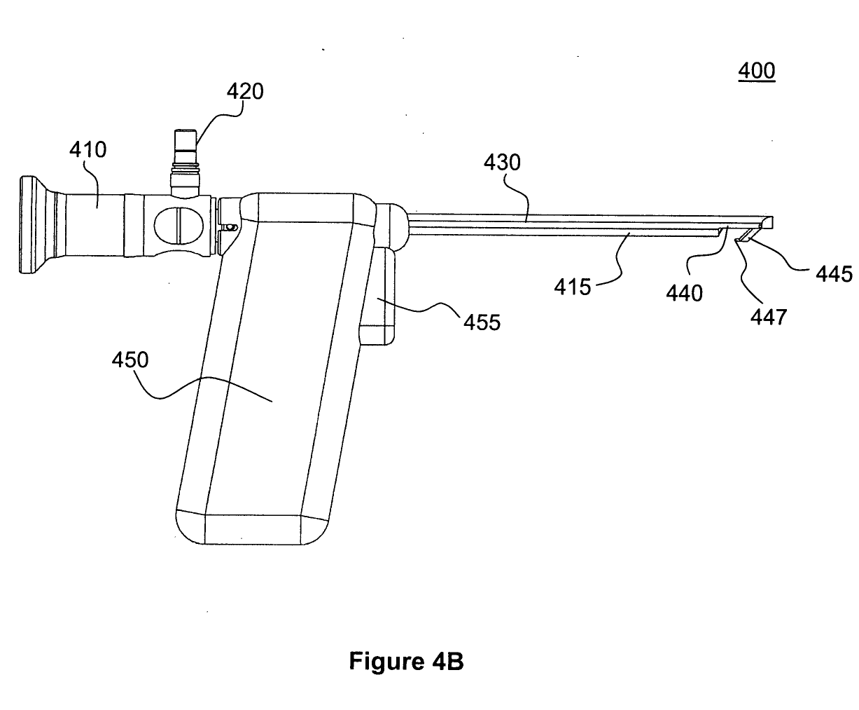

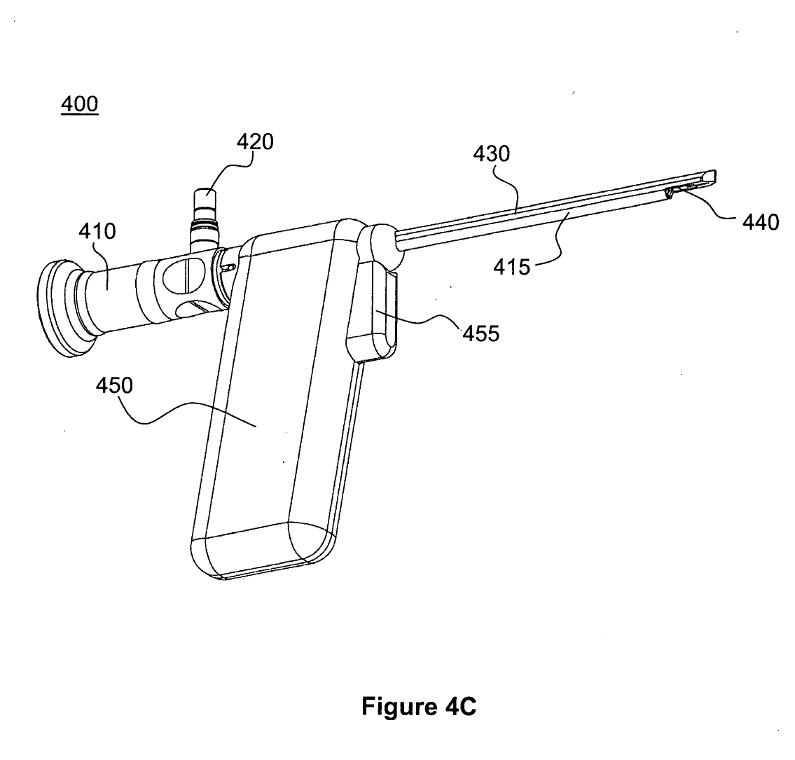

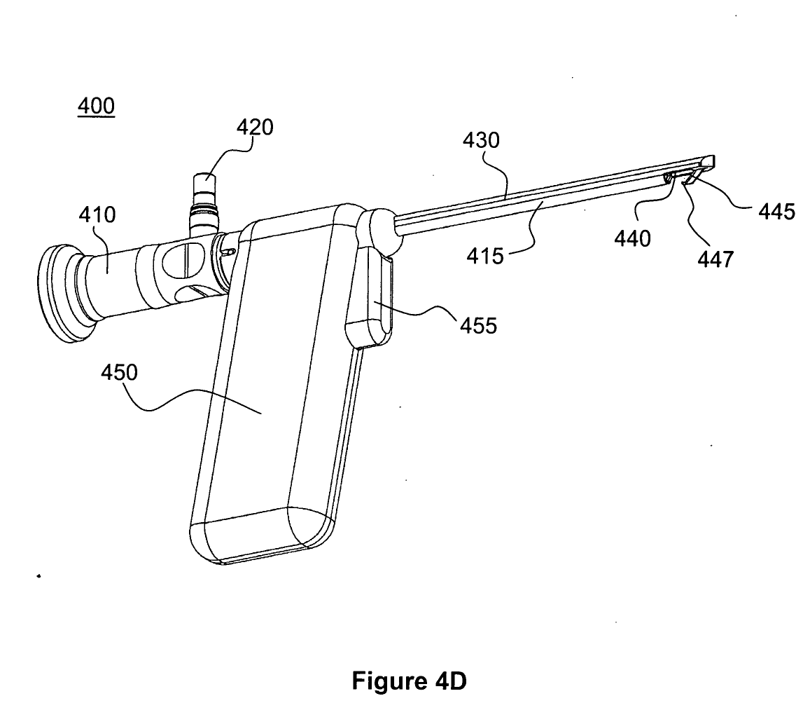

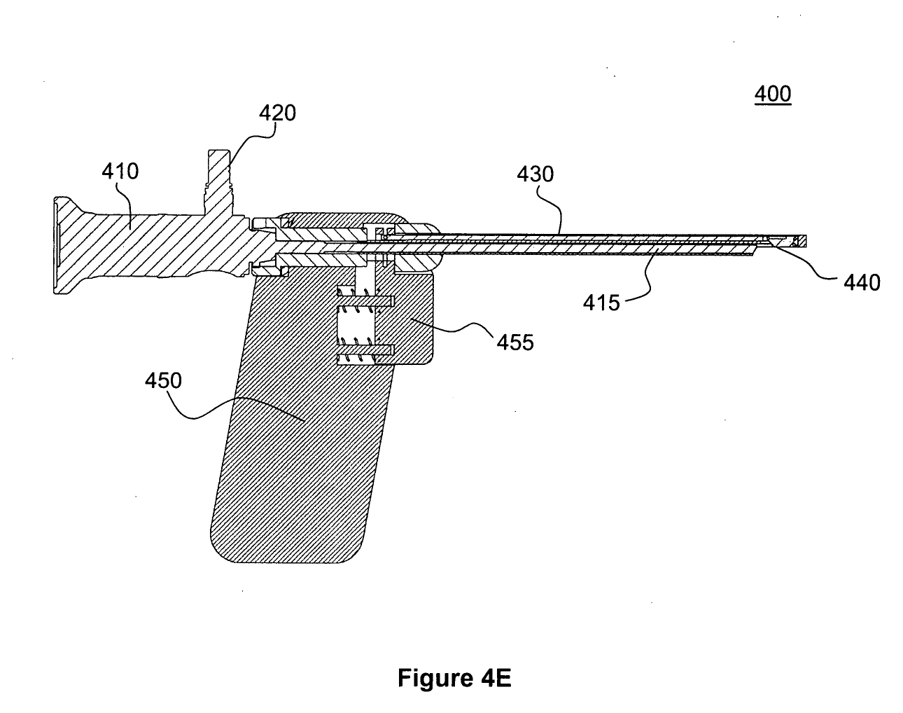

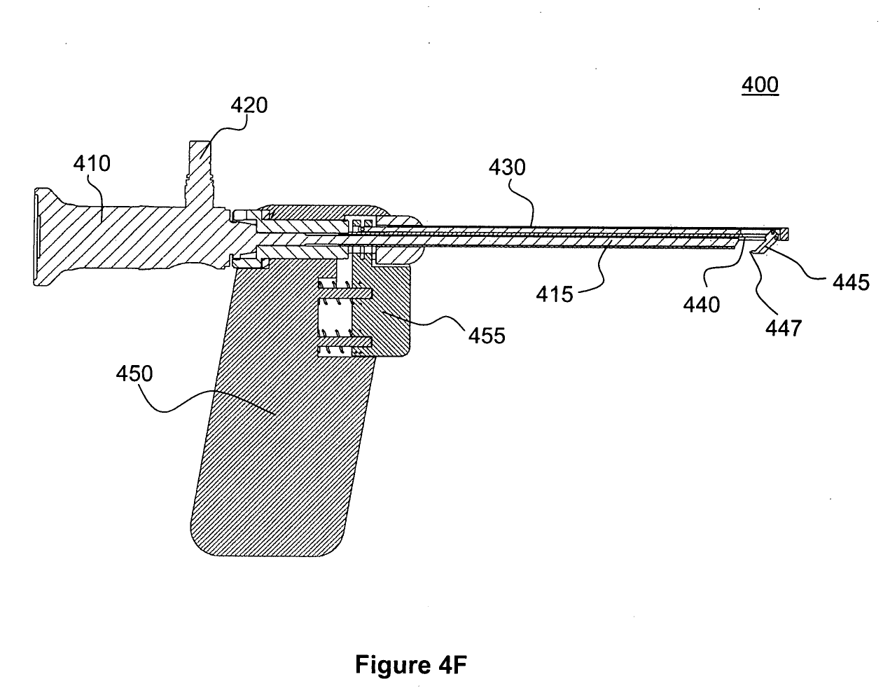

Then the cutting tool 400 is inserted into the incision and along the pocket 220. The cutting tool 400 is shown in FIGS. 4A (with cutting blade retracted) and 4B (with cutting blade extended). Corresponding perspective views of the cutting tool are shown in FIGS. 4C and 4D, respectively. Corresponding sectional views of the cutting tool are shown in FIGS. 4E and 4F, respectively. An endoscope body 410 supports an endoscopic tube 415 above which is mounted a probe 430, at the end of which is a retractable blade 445. The probe 430 contains the mechanism for retracting and extending the cutting blade 445. The probe 430 extends beyond the endoscopic tube 415, forming a recess 440. The recess 440 provides space for extension and retraction of the cutting blade 445, and also allows a clear field of view for a camera lens at the end of the endoscopic tube 415.

A pistol grip 450 has a trigger 455. When the trigger 455 is pulled toward the pistol grip 450, the blade 445 is extended from the recess 440. The pistol grip 450 and trigger 455 assembly are mounted on the endoscope so that the pistol grip 450 and trigger 455 assembly may be rotated around the axis of the endoscopic tube 415 and probe 430, so that the trigger 455 may be operated to retract and extend the cutting blade 445 without interfering with the hand or arm of the patient, while at the same time maintaining the orientation of the cutting blade 445 with respect to the sheath surrounding the tendons. The light for the endoscope is provided by fiber optic channels (not shown) within the endoscopic tube 415. The light source is provided through endoscopic control 420, which also channels the video signals coming from the camera lens.

A cross sectional representation of the cutting tool after insertion along the pocket 220 is shown as item 230 in FIG. 2A. The tissue clearing provided by the above described operation of the rasp tool improves the field of view provided by the camera lens at the end of endoscopic tube 415. Further improvement in the field of view is provided by controlling the light shown in the field of view. For example, ultraviolet light exposes features within the pocket 220 that would otherwise be obscured.

The cutting tool 400 is inserted into the pocket 220 until the depression 440 is beyond the edge of the upper sheath surface 240. The following description assumes that the incision has been made on the proximal side of the sheath, but those skilled in the art will understand that the incision could also be made on the distal side of the sheath. The probe 430 is oriented so that the tip of the probe is aligned so as to avoid nerve 210 and positioned on the sheath 240 within the pocket 220 as shown between the dotted lines 270. Trigger 455 is then actuated, extending blade 445 in a downward direction toward upper sheath surface 240. The blade 445 is shaped and extended at an angle so as to catch the sheath 240 on the cutting surface of the blade 445 as the cutting tool is pulled back out of the pocket. The cutting surface is located on the side of the extended blade 445 facing the pistol grip. The cutting blade 445, when extended, protrudes a certain distance outside the cross section formed by the probe 430 and endoscopic tube 415, enough to catch the edge of the sheath 240. The blade is guided by a smooth protrusion 447 on the lower edge of the blade 445, which operates so that the protrusion 447 remains beneath the sheath 240. As the cutting tool 400 is operated to cut the sheath 240 by pulling the tool out of the pocket, it may be necessary to reinsert the extended blade 445 into the line of the cut, so that a full release of the sheath is accomplished. A full release may be understood with reference to FIG. 1, where a cut begins at the edge of sheath 130 away from the incision and proceeds to the edge nearest the incision, between the dotted lines 140.

It should be noted that in a significant number of cases there may be a separate compartment around one of the wrist tendons, in which case an additional cut may be required to fully release the sheath enclosing both tendons. The need for an additional cut is usually confirmed by moving the thumb so as to observe movement of the tendons individually.

While the invention has been described in terms of a single preferred embodiment, those skilled in the art will recognize that the invention can be practiced with modification within the spirit and scope of the appended claims.

Having thus described my invention, what I claim as new and desire to secure by Letters Patent is as follows:

1. An apparatus for release of a tendon sheath, comprising:
a rasp tool having a body supporting a probe with a rasp surface at one end of the probe for removing soft tissue adhering to the tendon sheath after insertion of the probe into a pocket formed above the tendon sheath; and
an endoscopic cutting tool having a probe with a blade at one end, the blade being extendable by operation of a trigger after insertion of the probe into the pocket, the blade being operable to cut the tendon sheath by pulling the tool out of the pocket with the blade extended.

2. The apparatus of claim 1, wherein the rasp body and probe are fitted over an endoscope, a field of view for an endoscope camera being provided by a groove on the rasp surface.

3. The apparatus of claim 2, wherein the rasp body, rasp probe and rasp surface are of one piece, the one piece being removably attachable to the endoscope.

4. The apparatus of claim 1, wherein the trigger is part of a pistol grip assembly mounted on the endoscopic cutting tool, the assembly being able to rotate around the axis of the probe.

5. A rasp tool for use in release of tendon sheath, comprising:
a body;
a probe supported by the body;
a rasp surface at one end of the probe for removing soft tissue adhering to the tendon sheath after insertion of the probe into a pocket formed above the tendon sheath.

6. The tool of claim 5, wherein the rasp body and probe are fitted over an endoscope, a field of view for an endoscope camera being provided by a groove on the rasp surface.

7. The tool of claim 6, wherein the rasp body, rasp probe and rasp surface are of one piece, the one piece being removably attachable to the endoscope.

8. An endoscopic cutting tool for use in release of tendon sheath, comprising:
an endoscope having a body from which is extended an endoscope tube with a camera lens at a tube end away from the body;
a probe with a blade at one end, the probe being mounted on the endoscope tube so that the blade end extends beyond the end of the tube to allow for extension and retraction of the blade, the blade being extendable by operation of a trigger, the trigger being operated after insertion of the probe into a pocket formed above the tendon sheath, the blade being operable to cut the tendon sheath by pulling the tool out of the pocket with the blade extended.

9. An endoscopic cutting tool as in claim 8, wherein the trigger is part of a pistol grip assembly mounted on the endoscopic cutting tool, the assembly being able to rotate around the axis of the probe.

10. An endoscopic cutting tool as in claim 8, further comprising a light source and fiber optic channels for delivering light from the light source along the endoscopic tube to illuminate objects observable by the camera lens.

11. An endoscopic cutting tool as in claim 10, wherein the light source provides ultraviolet light.

12. A method for release of a tendon sheath, comprising the steps of:
making a lateral incision on one side of the tendon sheath, dissecting down to the tendon sheath, and dissecting across the tendon sheath to form a pocket above the tendon sheath;
using a rasp tool to remove soft tissue adhering to the tendon sheath, the rasp tool having a body supporting a probe with a rasp surface at one end of the probe, the soft tissue being removed after insertion of the rasp probe into the pocket formed above the tendon sheath; and
using an endoscopic cutting tool to release the tendon sheath, the endoscopic cutting tool having a probe with a blade at one end, the blade being extendable by operation of a trigger after insertion of the cutting tool probe into the pocket, the blade being operable to cut the tendon sheath by pulling the tool out of the pocket with the blade extended.

13. The method of claim 12, wherein the rasp body and probe are fitted over an endoscope, a field of view for an endoscope camera being provided by a groove on the rasp surface.

14. The method of claim 13, wherein the rasp body, rasp probe and rasp surface are of one piece, the one piece being removably attachable to the rasp endoscope.

15. The method of claim 12, wherein the trigger is part of a pistol grip assembly mounted on the endoscopic cutting tool, the assembly being able to rotate around the axis of the probe.

16. The method of claim 12, further comprising a light source and fiber optic channels for delivering light from the light source along the endoscopic tube to illuminate objects observable by the camera lens.

17. The method of claim 16, wherein the light source provides ultraviolet light.

18. The method of claim 12, wherein the incision is made on the distal side of the sheath.

19. The method of claim 12, wherein the incision is made on the proximal side of the sheath.
